(19) United States
(12) Patent Application Publication
(10) Pub. No.: US 2012/0258086 A1
(43) Pub. Date: Oct. 11, 2012

(54) PLATELET SOLUTION FOR USE IN JOINT SURGERY

(75) Inventors: Marc Long, Denville, NJ (US); Lin Song, Wayne, NJ (US)

(73) Assignee: HOWMEDICA OSTEONICS CORP., Mahwah, NJ (US)

(21) Appl. No.: 13/508,882

(22) PCT Filed: Nov. 11, 2010
(86) PCT No.: PCT/US2010/056341

§ 371 Date: Jun. 22, 2012

Related U.S. Application Data
(60) Provisional application No. 61/260,140, filed on Nov. 11, 2009.

(51) Int. Cl. A61K 35/14; A61K 38/18; A61P 19/02; A61K 38/19

(52) U.S. Cl. 424/93.72

(57) ABSTRACT

The invention is directed to a method of accelerating healing in a joint comprising administering a platelet composition to the joint.

### CROSS-REFERENCE TO RELATED APPLICATIONS

This application claims the benefit of the filing date of U.S. Provisional Patent Application No. 61/260,140, filed Nov. 11, 2009, the disclosure of which is hereby incorporated herein by reference.

### BACKGROUND

Different techniques are known in the art for promoting healing of joints following surgery. Platelets are one such agent for this purpose. Platelets secrete a number of factors including serotonin, fibronectin, ADP, thromboxane A, platelet factor 4, platelet-derived growth factor, and platelet activating factor. Release of these factors is believed to cause a chemotactic response which initiates the process of migration between endothelial cells. As more factors continue to be released from platelets as well as monocytes and macrophages, angiogenesis, osteogenesis and the formation of granulation tissue are promoted. Several platelet-based products are commercialized under the labeling of Platelet Rich Plasma (PRP), also referred to as Autologous Platelet Gel. The commercial products include the GPS® System (Biomet), Fibrinet (Cascade Medical), and SmartPrep (Harvest), which are described for use following arthroscopic repairs, such rotator cuff repair, meniscus repair, and ACL reconstruction. These PRP systems are disclosed as containing several growth factors, including vascular endothelial growth factor, platelet-derived growth factor, transforming growth factor-β, fibroblast growth factor and epidermal growth factor.

Iwata, et al., Muscle & Nerve 34(5):623-30 (2006), reports use of fibroblast growth factor-2 to enhance functional recovery of reinnervated muscle, e.g., following surgery.

Sanchez, et al., Med. Sci. Sports Exerc. 35(10):1648-52 (2003), reports the application of an autologous plasma rich in growth factors as being beneficial in restoring connective tissues following arthroscopic treatment of large, non-traumatic avulsions of articular cartilage in the knee.

Foster, et al., Am. J. Sports Med. 37(11):2259-72 (2009), is a review that reports on the use of Platelet-rich plasma for chronic tendinopathy, bone healing, acute ligamentous injuries, total knee arthroplasty, ACL reconstruction, acute achilles tendon repair, rotator cuff repair, acute cartilage and meniscus repair.

### SUMMARY OF THE INVENTION

An aspect of the present invention is directed to a method for expediting or accelerating recovery from joint surgery. The method entails administering to the joint, during the course of surgery, a composition containing platelets, allogeneic or autologous, with or without a pharmaceutically acceptable carrier. The composition may be administered directly to the joint during the course of, i.e., as an adjunct to surgery. Typically, the administration of the composition is the penultimate or last step in a surgical procedure, preferably an endoscopic, arthroscopic, or mini-open procedure, or prior to closure of the surgical incision. The administration may be part of the lavage step executed at the end of the surgery to replenish joint fluid. The administration may be directed into the joint space or the area directly surrounding the joint, such as the synovial fluid of the knee for example, or another joint.

Another aspect of the present invention is directed to a composition containing allogeneic or autologous platelets and a pharmaceutically acceptable carrier. The inventive compositions may further contain other therapeutic agents, such as growth factors, small biomolecules, and anti-inflammatory and anti-microbial agents, as well as inert ingredients, such as gelling agents, hydrophilic agents, surfactants and phospholipids. The composition may comprise elements naturally present in the synovial fluid, such as hyaluronans, glucosamine, chondroitin sulfate, aggrecans, and collagen. Thus, it may be used as a substitute for or complement to the joint fluid.

The compositions and methods of the present invention may provide for expedited recovery from joint surgery, e.g., via a catabolic effect to the joint environment, particularly where the underlying damage is due to injury, inflammation, and/or a disease or disorder such as osteoarthritis. The platelets not only deliver growth factors and produce an anabolic effect to the joint environment, they will also recruit the patient's own cells and proteins and produce proteins and other beneficial substances such as growth factors, all of which aid in tissue regeneration, repair, stabilization, lubrication, and pain reduction.

Although not intending to be bound by these theories, the mechanism of action of the platelets in the joint may involve the secretion of soluble factors and signals from these exogenous platelets. This secretion may in turn enable, accelerate, and/or enhance a positive biological process that benefits the recovery of the joint. These soluble factors may also play a chemotactic role in attracting circulating cells and/or proteins in the joint or in the vicinity of the joint.

### DESCRIPTION OF THE FIGURE

FIG. 1 is a bar graph showing growth of human bone marrow mesenchymal stem cells (expressed in units of optical density) in vitro, as a function of comparative culture conditions, including a representative embodiment of the present invention.

### DETAILED DESCRIPTION

Autologous and allogeneic blood types are sources of platelets suitable for use in the present invention. In a preferred embodiment, these platelets are autologous. Platelets may be separated and harvested from blood using a variety of methods. Separation methods include centrifugation, filtration, antibody labeling, magnetic separation, peptide-based separation, agglutination of red blood cells, ballooning of the platelets to increase their size, or a combination thereof.

The platelets for use in the present invention may further be manipulated prior to administration to enhance their performance and efficacy in the joint environment. For example, the platelets may be “primed” or pre-challenged to simulate the hostile joint environment in which they will be placed. As the joint is likely to be exposed to inflammatory processes, one preferred embodiment entails a pre-challenge of the platelets in an inflammatory environment, such as in the presence of IL-1β, IL-1β converting enzyme, or TNF-α, or other inflammatory cytokines or compounds. In another preferred embodiment, the platelets are pre-challenged in the presence of enzymes which are commonly present in operated joints or diseased joints. Such enzymes include proteolytic enzymes such as aggrecanase, collagenase, caspases, and matrix metalloproteinases (MMPs).

In another embodiment, the platelets are treated so that they can preferentially target specific tissue or tissues, a process referred to as homing. The treatment may include attaching to the platelets a specific peptide, or peptides, or other molecule having a segment that binds to the platelets, and another active segment which specifically binds to specific tissue(s) in the joint, such as synovial tissue, articulating cartilage and/or fibrocartilage (meniscus) tissue, ligament and/or tendon and/or muscle tissue (ACL, PCL, rotator cuff, biceps, etc.).

The platelets may be fully or partially activated prior to insertion into the joint space. Activation of the platelets may be rapid, as for instance with the use of an activation factor such as thrombin or bovine thrombin. Activation may be less rapid and partial, as for instance with the use of calcium chloride or other non-thrombin-based platelet activator, such as collagen. Partial activation is intended to produce a slow release of growth factors and cytokines from the platelets.

The activation factor may be added to the platelets prior to, during, or after formulation with the carrier. A different activation factor may be added to the platelets prior to the addition to the carrier and added or formulated into the carrier.

The selection of a suitable pharmaceutically acceptable carrier is within the skill of the ordinary artisan. Representative examples include plasma (autologous or allogeneic), serum (autologous or allogeneic), water for injection, fibrin, fibrinogen, hyaluronan, chemically modified hyaluronan, saline, phosphate buffered saline, chondroitin sulfate, glucosamine, mannosamine, proteoglycan, proteoglycan fragments, chitin, chitosan, or other polysaccharide or polymer material, and combinations thereof.

Suitable gel and/or gel-forming substances may also be included in the composition. The gel and/or gel-forming substance may contain an adhesive material such as fibrin, collagen or a transglutaminase system, to adhere the gel or formed gel to the tissues surrounding the site of administration. Suitable gels and gel-forming substances include biologically-based polymers such as a collagen solution or fibrous suspension, hyaluronan, chemically modified hyaluronan, chitosan (hydrolysed chitin), and synthetic polymers such as a photopolymerizable end-capped block copolymer of poly(ethylene oxide) and an α-hydroxy acid. The compositions may further contain surfactants (e.g., lubricin), lipids (e.g., glycerols) and phospholipids or surface active phospholipids (SAPL) (e.g., DPPC, PLPC, POPC, SLPC, and combinations thereof), or combinations thereof.

The carrier may be formulated in such a way that the activation of the platelet and/or the release of growth factors, cytokines, and proteins from the platelet. The carrier may be cross-linked after the addition of platelets. The carrier may be a self-cross linking or a self-polymerizing formulation. The carrier may be added to the platelets or may already be present with the platelets when extracted from the blood. In a preferred embodiment, this carrier is fibrin or fibrinogen.

The compositions may further include cells. Cells may be autologous, allogenic, xenogenic, or a combination thereof. Suitable types of cells include, for example, stem cells, mesenchymal stem cells, and/or progenitor cells, including cells derived from bone marrow, synovial fluid, synovium, placenta, umbilical cord, skin, muscle, and fat/adipose tissue. Cells may also be differentiated cells including, for example, chondrocytes, tenocytes, osteoblasts, and synoviocytes. The composition may include one type of cells or a combination of two or more cell types.

The compositions may further include other, non-cellular therapeutically beneficial agents such as growth factors (e.g., TGF-β, EGF, FGF, IGF-1 BMP-7 and OP-1, etc.), glycosaminoglycans (GAGs) (e.g., aggrecan, decorin, biglycan and fibromodulin), chemokines and cytokines (e.g., interleukins and interferons) and hydrophilic compounds (e.g., polylysine, chitosan and hyaluronan). Extracellular matrix molecules that bind to growth factors, e.g., heparan sulfate proteoglycans, may advantageously be added to serve as a reservoir for the factors.

Accordingly, the compositions of the present invention may be administered to affected and surgically repaired joints including knee joints, hip joints, shoulder joints, elbow joints, ankle joints, tarsal and metatarsal joints, wrist joints, spine, carpal and metacarpal joints, and the temporal mandibular joint. The joint may be osteoarthritic or otherwise exhibits one or more symptoms (characteristics) of inflammation.

The platelets are administered in an amount effective to expedite or accelerate recovery of the joint from surgery. Normal platelet counts in blood range from about 150,000/μL, to about 350,000/μL. Platelets are not typically present in the natural joint space and joint fluid. Platelets may be delivered in an amount between about 1/μL, to about 10,000,000/μL, preferably between about 100,000/μL, to about 5,000,000/μL, most preferably between about 1,000,000/μL, to about 5,000,000/μL. The platelets are typically delivered in a volume of about 1 to about 10 ml, via any medically acceptable device for delivering fluids to open surgical areas or wounds. The number of platelets is dependent upon a variety of factors, including the age, weight, and sex of the patient, the tissue or tissues being targeted for healing, the extent and severity of the damage or injury to the joint, or of the disease affecting the joint, the degree of exudation within the joint, the joint space, and other anatomical characteristics that might influence the delivery.

In another embodiment, the platelets are delivered in a platelet-rich plasma, with a platelet concentration of at least about 100,000/μL in 1 to 10 mL of plasma, and preferably at least about 1,000,000/μL in 1 to 10 mL of plasma. Platelet-rich plasma may contain a 2- to 10-fold increase in growth factor concentrations.

In another embodiment, the platelets are delivered in a platelet-poor plasma, with a platelet concentration lower than that of normal blood. The platelet-poor plasma may be collected from the platelet concentration process and delivered into the joint space for additional biological enhancement.

The platelets for use in the present invention may be administered in the joint via a variety of techniques. In a preferred embodiment platelet solution is added to last bag for last step lavage at the end of an arthroscopic intervention. In another preferred embodiment, platelet solution is administered by pouring, squirting, spraying, and/or flowing in the joint space prior to closure of the joint. In another preferred embodiment, the platelet solution is injected prior to closing the joint or after the joint is closed at the end of the surgical intervention.

The timing of the administration during the course of a surgery is not critical, but is typically performed as the penultimate or last step prior to closure of the surgical opening.

Administration of the platelet solution may be performed once or in series over the course of days, weeks, or months after surgery.

The present invention will now be described in terms of the following non-limiting example.

### Example 1

### PRP Promotes Stem Cell Proliferation

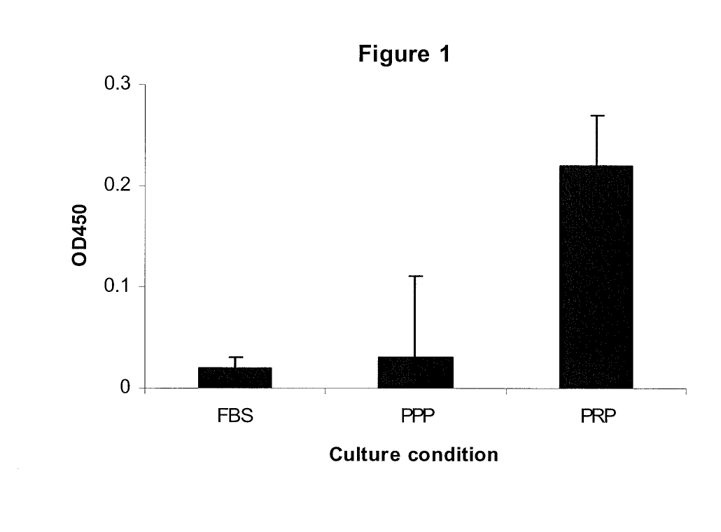

Human whole blood was collected into BD Vacutainer CPT tubes and spun at 1500 RCM for 20 min. The top layer containing an inventive composition, referred to in this example as platelet-rich plasma (PRP) was collected and used within an hour. To prepare platelet-poor plasma (PPP), PRP was spun at 2000 RCM for 20 min to remove platelets. Human bone marrow mesenchymal stem cells (MSCs) were expanded and used at passage 3. For proliferation, hMSCs were cultured in DMEM containing 1% antibiotics and 10% of FBS, PPP or PRP. At day 4, cell numbers were quantified using Cell Counting Kit-8 (CCK-8). As shown in FIG. 1, PRP induced higher MSC proliferation than FBS or PPP. Thus, the present invention may achieve at least one unexpected result in terms of stimulating proliferation of endogenous or exogenously added (autologous or allogeneic) stem cells present in the joint environment, thus accelerating the healing process.

All publications cited in the specification, both patent publications and non-patent publications are indicative of the level of skill of those skilled in the art to which this invention pertains. Any publication not already incorporated by reference herein is herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of enhancing recovery from joint surgery, comprising administering to the joint during surgery a composition comprising platelets in an amount effective to enhance recovery, and a pharmaceutically acceptable carrier.

2. The method of claim 1, wherein said platelets are autologous.

3. The method of claim 1, wherein said platelets are allogeneic.

4. The method of claim 1, wherein prior to being administered, said cells are homed to specifically bind one or more joint tissues, including synovial tissue, joint capsule, articulating cartilage, fibrocartilage (meniscus) tissue, ligament or tendon tissue.

5. The method of claim 1, wherein said carrier comprises plasma, serum, water for injection, fibrin, fibrinogen, hyaluronan, chemically modified hyaluronan, saline, phosphate buffered saline, chondroitin sulfate, glucosamine, mannosamine, proteoglycan, proteoglycan fragments, chitin, chitosan, or mixtures thereof.

6. The method of claim 1, wherein said composition further comprises a gelling agent, a hydrophilic agent, a surfactant, a lipid, a phospholipid, a surface-active phospholipid, or mixtures thereof.

7. The method of claim 1, wherein said composition further comprises a non-cellular therapeutic agent.

8. The method of claim 7, wherein said agent comprises a growth factor, cytokine, chemokine, hydrophilic compound, or an extracellular matrix compound.

9. The method of claim 7, wherein said agent is BMP-7.

10. The method of claim 1, wherein said joint is the knee, hip, shoulder, elbow, ankle, tarsal or metatarsal, wrist, spine, carpal or metacarpal, or temporal mandibular joint.

11. The method of claim 1, wherein the surgery is arthroscopic.

12. The method of claim 1, wherein the surgery is endoscopic.

13. The method of claim 1, wherein said composition is administered via injection prior to closing the joint or after the joint is closed.

14. The method of claim 1, wherein the joint is osteoarthritic.

15. The method of claim 1, wherein the joint exhibits one or more symptoms of inflammation.

16. The method of claim 1, wherein the platelets are co-administered with autologous or allogeneic stem cells.
